(19) United States
(12) Patent Application Publication
(10) Pub. No.: US 2025/0086784 A1
(43) Pub. Date: Mar. 13, 2025

(54) Biological Age and Survival Risk Determination from Imaging Biomarkers

(71) Applicant: Wisconsin Alumni Research Foundation, Madison, WI (US)

(72) Inventors: Perry Pickhardt, Madison, WI (US); John Garrett, Madison, WI (US); Michael Kattan, Cleveland Heights, OH (US)

(21) Appl. No.: 18/243,833

(22) Filed: Sep. 8, 2023

(51) Int. Cl. G06T 7/00; G16H 10/60; G16H 50/30

(52) U.S. Cl. CPC G06T 7/0012; G16H 10/60; G16H 50/30; G06T 2207/10081; G06T 2207/30004

(57) ABSTRACT

The assessment of multiple body composition measures from CT or other volumetric scans can be combined to provide a panoptic understanding of an individual's health made practical by analysis of CT images obtained for other purposes through automatic techniques, or by intended or planned CT screening.

### STATEMENT REGARDING FEDERALLY SPONSORED RESEARCH OR DEVELOPMENT

—

### CROSS REFERENCE TO RELATED APPLICATION

—

### BACKGROUND OF THE INVENTION

The present invention relates to medical imaging systems and in particular to a system using either opportunistic or intended medical imaging data, for example, from a CT scanner, for cost-efficient, panoptic health assessment.

The availability of sophisticated volumetric medical imaging tools such as computed tomography (CT) has led to the development of a variety of analysis tools that can receive volumetric image data and assess body composition measures such as: bone mineral density, visceral and subcutaneous fat, skeletal muscle, aortic and coronary calcific plaque, and hepatosplenic measures.

While these measures can provide helpful clinical information, the availability of lower-cost alternatives for specific screening indications (e.g., ultrasound monitoring of plaque, dedicated DEXA machines for bone scanning, etc.) has generally precluded the use of such volumetric data for routine screening.

### SUMMARY OF THE INVENTION

The present invention combines multiple body composition measurements into a single measurement to provide greater insight into an individual's health than can be obtained from these body composition measurements in isolation. The ability to extract multiple measurements from a single volumetric medical scan using automated tools makes such a combined measurement practical, for example, opportunistically leveraging volumetric medical scans that are obtained for other clinical indications.

In one embodiment, the invention provides a health screening system including a set of computerized image analysis tools receiving slice-image medical data of the patient to provide quantitative measurements of a set of body composition measurements related to clinical risk factors. A model receives the different body composition measurements to provide a health assessment value combining the different body composition measurements into a health assessment value indicating the health of the patient which is provided to an output presenting the health assessment value for review.

It is thus a feature of at least one embodiment of the invention to capture the synergy in a combination of automated body composition measurements practically derived from a single slice image set.

The health assessment value may be an age value.

It is thus a feature of at least one embodiment of the invention to provide a health assessment value that is intuitively understandable to patients.

The model may provide an empirically derived survival probability and wherein the age value may be obtained by matching the survival probability obtained from the model to a second distinct model relating survival probability to chronological age.

It is thus a feature of at least one embodiment of the invention to provide a method of converting readily derived survivability to biological age.

The second model may model input parameters independent of body composition information derived from slice imaging, for example, including parameters selected from the group of sex, race, and smoking history.

It is thus a feature of at least one embodiment of the invention to make use of pre-existing actuarial data to inform or compare the measurement of biological age.

The output may further provide relative significance of the body composition measurements in influencing the health assessment value.

It is thus a feature of at least one embodiment of the invention to provide a health assessment that is both transparent to the extent that it exposes the influences of the health assessment and actionable to the extent that it may point to patient treatment plans.

The computerized analysis image tools may output physical measurements.

It is thus a feature of at least one embodiment of the invention to employ “explainable” automated models as workings that can be reviewed and even validated by a human.

In this regard, the output may further provide images depicting the physical measurements.

It is thus a feature of at least one embodiment of the invention to permit oversight of the health assessment evaluation by a clinician.

The body composition measurements may be selected from the group consisting of biomarkers such as (but not limited to): bone density, fat classification, area, and density, muscle bulk and density, liver volume and density, spleen volume, and aortic plaque burden.

It is thus a feature of at least one embodiment of the invention to permit the use of a rich set of existing body composition measurement tools.

The volumetric image data is computed tomography data and may, for example, be abdominal or thoracic imaging.

It is thus a feature of at least one embodiment of the invention to use an image modality and region that is both common, for example, for virtual colonoscopies or lung cancer screening, and that is suitable for making a robust variety of body composition measurements.

These particular objects and advantages may apply to only some embodiments falling within the claims and thus do not define the scope of the invention.

### BRIEF DESCRIPTION OF THE DRAWINGS

FIG. 1 is a simplified diagram of a CT system suitable for collecting and processing volumetric data according to the present invention according to a stored program.

FIG. 2 is a flowchart of the program of FIG. 1 showing various steps in the processing; and

FIG. 3 is an example output display of data generated by the present invention.

### DETAILED DESCRIPTION OF THE PREFERRED EMBODIMENT

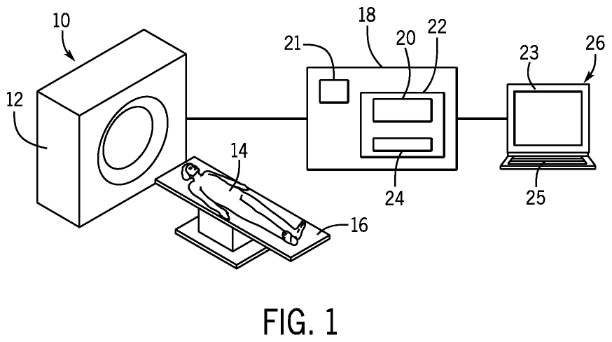

Referring now to FIG. 1, a medical imaging machine such as a computed tomography (CT) machine 10 may provide a gantry 12, for example, holding an opposed rotating x-ray source and detector for collecting tomographic data on a patient 14 as the patient is moved through the x-ray beam on a patient table 16. The volume data 20 may be stored in electronic memory 22 of a processing system 18 having one or more processors 27 executing a stored program 24 for acquiring and reconstructing the tomographic data into volume data 20.

Generally the processing system 18 may communicate with a user terminal 26, for example, having a graphic display 23 and an input device 25 such a keyboard or mouse or the like. More generally, the processing system 18 may include the processor used by the CT machine for reconstruction and CT acquisition tasks and/or one or more remote processors, for example, communicating with the processing system 18 to obtain volume data 20 via a DICOM server or the like.

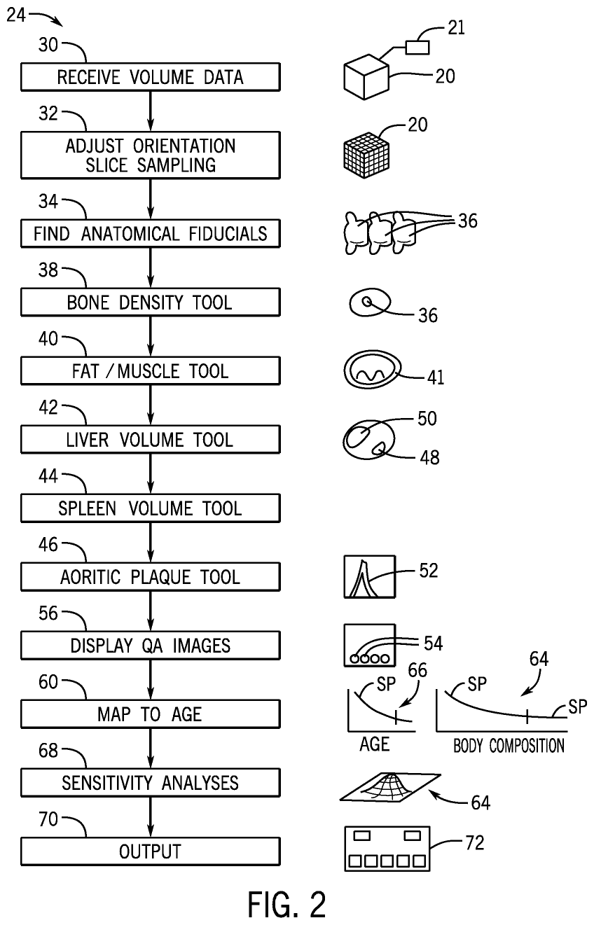

Referring now to FIG. 2, the program 24 may operate to receive the volume data 20 acquired on the CT machine 10 per process block 30. In a preferred embodiment, the volume data 20 may be taken from an abdominal scan of the patient 14, for example, during a colorectal cancer screening or other clinical purpose and can be used by the present invention to develop a separate health assessment. The volume data 20 describes a set of voxels spaced over three dimensions, each denoting an x-ray attenuation value, for example, in Hounsfield numbers and stored in a DICOM files identified to a given series. The volume data 20 may be associated with other patient bibliographic data 21, for example, with a linkage with an electronic health record.

At process block 32, the volume data 20 may be preprocessed to a standard form assisting in automatic assessment. This preprocessing includes ensuring that the patient orientation observed a standard Left-Anterior-Superior (LAS) voxel ordering and equalizing sampling or resampling to a uniform 3×3 mm sampling and thickness. At this step CT number normalization (removing any vendor HU offset) is also performed as well as conversion to a single NIFTI file providing a standard format more amenable to data analysis.

At succeeding process block 34, reference anatomical fiducial points are identified for the subsequent body composition measurements in the form of the level-specific indicators, such as the first and third lumbar vertebral bodies 36 (L1 and L3 levels). These reference points ensure that measurements being taken of the patient 14 are at similar locations in the abdomen (or chest) for earlier and later assessments to provide more consistent longitudinal assessments and to better match the location of the data of the given patient 14 with data used in the development of the various tools and models to be described below.

The localization of the vertebral bodies may be automatically performed using a convolutional neural network (CNN) based an Unsupervised Body Part Regression (UBR) approach, for example, as described in Ke Yan L L, Ronald M. Summers, Unsupervised Body Part Regression via Spatially Self-ordering Convolutional Neural Networks, 2018 IEEE 15th International Symposium on Biomedical Imaging (ISBI 2018), Washington, DC, USA, 2018, pp. 1022-1025, DOI: 10.1109/ISBI.2018.8363745; and Ke Yan X W, Le Lu, Ling Zhang, Adam Harrison, Mohammadhad Bagheri, Ronald Summers, Deep Lesion Graphs in the Wild: Relationship Learning and Organization of Significant Radiology Image Findings in a Diverse Large-scale Lesion Database, (https://arxiv.org/abs/1711.10535). This deep learning model may be implemented in Caffe described in Jia Y, Shelhamer E, Donahue J, et al. Caffe: Convolutional Architecture for Fast Feature Embedding, Proceedings of the 22nd ACM International Conference on Multimedia, Orlando, Florida, USA: Association for Computing Machinery; 2014:675-678 and provides predicted levels for the twelfth thoracic (T12) through the fourth lumbar (L4) vertebral bodies as slice numbers. Once these lumbar levels are estimated, the remaining tools to now be described can be run in any order.

At process block 38, a surrogate bone mineral density measurement is made in CT number values (in Hounsfield Units or HU) at the L1 level. This measurement is made via an automatically placed ROI in the trabecular bone of the L1 vertebral body (identified as described above) using deep learning techniques described in Summers R M, Baccher N, Yao J, et al., Feasibility of simultaneous computed tomographic colonography and fully automated bone mineral densitometry in a single examination, J Comput Assist Tomogr 2011; 35 (2): 212-6, DOI: 10.1097/RCT.0b013c3182032537.

At process block 40, automated segmentation of abdominal fat 41 at the L1 and L3 levels are performed as described in Lee S J, Liu J, Yao J, Kanarek A, Summers R M, Pickhardt P J., Fully automated segmentation and quantification of visceral and subcutaneous fat at abdominal CT: application to a longitudinal adult screening cohort, The British journal of radiology 2018; 91 (1089): 20170968, DOI: 10.1259/bjr.20170968. This semantic segmentation classifies all voxels in the selected slices as either visceral fat, subcutaneous fat, or neither using fuzzy c-means to cluster fatty tissues and active contour models to separate subcutaneous and visceral fat per Yao J H, Sussman D L, Summers R M. Fully Automated Adipose Tissue Measurement on Abdominal C T, in: Weaver J B, Molthen R C, eds. Medical Imaging 2011: Biomedical Applications in Molecular, Structural, and Functional Imaging, Bellingham: Spic-Int Soc Optical Engineering; 2011.

From this segmented image, the areas of subcutaneous adipose tissue (SAT), visceral adipose tissue (VAT), and total adipose tissue (TAT) are calculated (in cm3). In addition, the ratio of VAT to SAT areas (VSR) is calculated. Similarly, an automated segmentation of abdominal muscle is performed at these same levels following the teachings of Burns J E, Yao J, Chalhoub D, Chen J J, Summers R M, A Machine Learning Algorithm to Estimate Sarcopenia on Abdominal C T, Academic radiology 2020; 27 (3): 311-320. DOI: 10.1016/j.acra.2019.03.011; Pickhardt P J, Perez A A, Garrett J W, Graffy P M, Zea R, Summers R M, Fully-Automated Deep Learning Tool for Sarcopenia Assessment on CT: L1 Versus L3 Vertebral Level Muscle Measurements for Opportunistic Prediction of Adverse Clinical Outcomes, AJR American journal of roentgenology 2021, DOI: 10.2214/AJR.21.26486; Ronneberger O, Fischer P, Brox T., U-Net: Convolutional Networks for Biomedical Image Segmentation, Lecture Notes in Computer Science: Springer International Publishing; 2015:234-241; and Pickhardt P J, Perez A A, Garrett J W, Graffy P M, Zea R, Summers R M., Fully Automated Deep Learning Tool for Sarcopenia Assessment on CT: L1 Versus L3 Vertebral Level Muscle Measurements for Opportunistic Prediction of Adverse Clinical Outcomes, AJR American journal of roentgenology 2021:1-8. DOI: 10.2214/ajr.21.26486. From this segmentation both the area of muscle as well as the mean CT number are calculated.

Per process block 42, 44, and 46 several volumetric segmentations are performed. These include two organs: the liver 50 and spleen 48, as well as aortic calcification of the aorta 52. The liver and spleen segmentations are performed using a modified 3D U-Net and automated error detection and refinement according to Isensee F, Kickingereder P, Wick W, Bendszus M, Maier-Hein K H, Brain tumor segmentation and radiomics survival prediction: Contribution to the BRATS 2017 challenge, Lecture Notes in Computer Science (including subseries Lecture Notes in Artificial Intelligence and Lecture Notes in Bioinformatics) 2018:287-297 and Özgün Çiçek AA, Soeren S. Lienkamp, Thomas Brox, Olaf Ronneberger. 3D U-Net: Learning Dense Volumetric Segmentation from Sparse Annotation. Medical Image Computing and Computer-Assisted Intervention-MICCAI 2016; and Sandfort V, Yan K, Pickhardt P J, Summers R M. Data augmentation using generative adversarial networks (CycleGAN) to improve generalizability in CT segmentation tasks, Sci Rep 2019; 9 (1): 16884. (In eng), DOI: 10.1038/s41598-019-52737-x.

For each segmented organ, the overall volume and mean CT number is measured. Additionally, aortic calcified plaques are automatically segmented using a modified U-Net per Isensee F, Kickingereder P, Wick W, Bendszus M, Maier-Hein K H. Brain tumor segmentation and radiomics survival prediction: Contribution to the BRATS 2017 challenge. Lecture Notes in Computer Science (including subseries Lecture Notes in Artificial Intelligence and Lecture Notes in Bioinformatics) 2018:287-297; and4 Özgün Çiçek AA, Soeren S. Lienkamp, Thomas Brox, Olaf Ronneberger. 3D U-Net: Learning Dense Volumetric Segmentation from Sparse Annotation. Medical Image, and Summers R M, Elton D C, Lee S, et al. Atherosclerotic Plaque Burden on Abdominal CT: Automated Assessment With Deep Learning on Noncontrast and Contrast-enhanced Scans. Acad Radiol 2020 (In eng). DOI: 10.1016/j.acra.2020.08.022. The volume of plaque and Agatston score at each slice location is provided by the tool and the sum of the scores from the aortic hiatus to the bifurcation are calculated.

Each segmentation (except for fat, which is only calculated at a single vertebral level, for example, in a single slice of the CT volume) is returned as a 3D DICOM series along with the normalized and resampled CT volume used for calculations. Each of these DICOM volumes shares a frame of reference UID and is perfectly co-registered for review in PACS or another DICOM viewer.

Each segmentation may be displayed as 2D, MIP, or 3D volume-rendered color-coded quality assessment image 54 (or manipulable 3-D image) per process block 56. Significantly, the automatic segmentations employed by the present invention employ understandable or “explainable” quantitative AI duplicating measurements that could be made or reviewed by a radiologist but are impractical and time prohibitive in the present situation. In this way, the invention may avoid opaque “black box” radiomics that may deter clinical acceptance from clinicians and patients. Because both the measured values and quality assessment images 54 used to generate those metrics will be provided, it is easy to identify cases where the tools fail and bolster confidence in the accepted measured values.

Various body composition measurements that may be made at process blocks 38, 40, 42, 44, and 46 are summarized in Table I below:

TABLE I

| Vertebral | Bone | Fat | Muscle | Liver | Spleen | Aortic Plaque |
|---|---|---|---|---|---|---|
| Levels | Metrics | Metrics | Metrics | Metrics | Metrics | Metrics |
| T12-L4 Slice | L1 Trabecular | L1/L3 VAT, | L1/L3 Area | Volume and | Volume and | Agatston score |
| numbers | CT Number | SAT, TAT, V/S | and CT | CT Number | CT Number | |
| | | Ratio | Number | | | |

Referring now to process block 60, the body composition values obtained with the above tools may be applied to a model 64 providing survival probabilities as a function of the body composition values. This model 64 may be constructed, for example, using the Cox proportional hazards regression, generally understood in the art, applied to a large set (over 10,000) of similar body composition measurements for other patients. The set of body composition measurements will be linked to mortality data indicating either a relative date of death of the patient 14 after acquisition of the volume data 20 or the current age of the patient 14 after this acquisition if the patient is alive.

As noted, the model of process blocks 60 provides a multidimensional surface (represented as a single dimension in FIG. 2) providing a survival probability SP as a function of these various body composition values. A second model 66 is also provided, also constructed using the Cox proportional hazards regression but based on data relating standard biological predictors such as age, sex, race, and smoking history, parameters that can be derived independently from data obtained from imaging and more specifically different from the body composition measurements informing the first model. Ten-year predicted survival probabilities are obtained for the patient 14 using the first model and this survival probability is iteratively applied to move a patient's age applied to the second model until the survival probability of the second model 66 matches the survival probability of the first model 64. The resulting age in the second model 66 then becomes a predicted biological age obtained from the present invention.

At process block 68, a sensitivity analysis may be performed around the iterated survival probability value of the first model 64 to determine the sensitivity of that prediction to the various body composition measurements provided to that first model 64, for example, indicating that changes in particular body composition measurement would be expected to produce the greatest change in survival probability. Such sensitivity may, for example, be obtained by obtaining a gradient of the model 64 at the point of prediction.

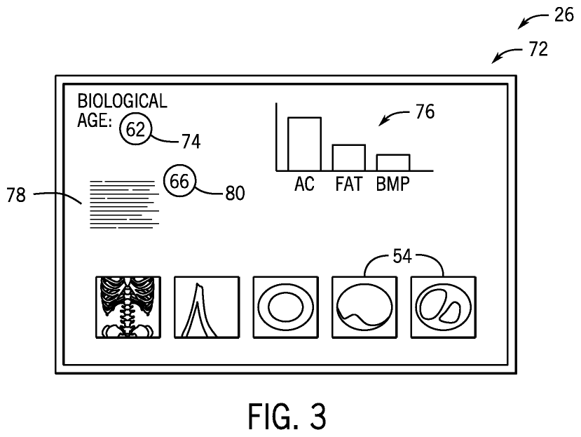

Referring now also to FIGS. 2 and 3, at process block 70, the collected data may be output as an output screen 72 on graphic display 23 presenting the computed biological age 74 from process block 60, the quality assessment images 54 from process block 56, and an indication of the relative sensitivity of the biological age 74 to the most significant body composition measurements affecting biological age 74. One such presentation may be a bar chart 76 showing relative significance, in this case indicating that aortic calcification is the most significant contributor followed by fat and bone mineral density.

The output screen 72 may also provide biographical information 78 about the patient as well as the patient's actual chronological age 80.

While combining body composition measurements provides a simple to understand health metric inherent in a single number, the combination is believed to also provide a more accurate health assessment value to the extent that the influence on health of different body composition measurements can offset each other, for example, poor measurements in one body composition category may be offset by good measurements in another category. Further, the tool has potential to exploit previously unknown interrelationships between these body composition measurements that can potentially increase the accuracy of the prediction.

The inventors contemplate that the present invention can also be used to provide a teaching set generally relating volumetric data 20 to biological age 62 for a large number of people that may have utility in training later artificial intelligence systems to operate directly on volume data 20 to produce biological age 74.

Certain terminology is used herein for purposes of reference only, and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

References to “a processor” and “a computer” or can be understood to include one or more integrated circuit processing devices that can communicate in a stand-alone and/or a distributed environment(s), and can thus be configured to communicate via wired or wireless communications with other processors, where such one or more processor can be configured to operate on one or more processor-controlled devices that can be similar or different devices. Furthermore, references to memory, unless otherwise specified, can include one or more processor-readable and accessible memory elements and/or components that can be internal to the processor-controlled device, external to the processor-controlled device, and can be accessed via a wired or wireless network.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties.

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What we claim is:

1. A health screening system comprising:
a set of computerized image analysis tools receiving slice-image medical data of a patient to provide a set of different body composition measurements related to clinical risk factors;
a model receiving the different body composition measurements to provide a health assessment value combining the different body composition measurements and indicating health of the patient; and
an output presenting the health assessment value for review.

2. The health screening system of claim 1 wherein the health assessment value is an age value.

3. The health screening system of claim 2 wherein the model provides an empirically derived survival probability and wherein the age value matches the survival probability obtained from the model to a second distinct model relating survival probability to chronological age.

4. The health screening system of claim 3 wherein the second model models input parameters independent of body composition information derived from slice image medical data.

5. The health screening system of claim 4 wherein the input parameters of the second model include characteristics of the patient selected from the group of sex, race, and smoking history.

6. The health screening system of claim 1 wherein the output further provides relative significance of the body composition measurements in influencing the health assessment value.

7. The health screening system of claim 1 wherein the computerized analysis image tools output physical measurements.

8. The health screening system of claim 7 wherein the output further provides images depicting the physical measurements.

9. The health screening system of claim 1 wherein the body composition measurements are selected from the group consisting of: bone density, fat proportion, muscle proportion, liver volume, spleen volume, and aortic plaque.

10. The health screening system of claim 1 wherein the slice image medical data is computed tomography data.

11. The health screening system of claim 10 wherein the slice image data is abdominal image data.

12. A method of assessing a biological age comprising:
(a) obtaining slice image medical data for a patient;
(b) applying a set of computerized image analysis tools to the slice image medical data to provide a set of different body composition measurements related to clinical risk factors;
(c) applying the set of different body composition measurements to a computerized model combining the different body composition measurements into a health assessment value; and
(d) outputting the health assessment value for review.

13. The method of claim 12 wherein the health assessment value is an age value.

14. The method of claim 13 wherein the model provides an empirically derived survival probability and wherein the age value matches the survival probability obtained from the model to a second distinct model relating survival probability to chronological age.

15. The method of claim 14 wherein the second model models input parameters independent of body composition information derived from slice image imaging.

16. The method of claim 15 wherein the input parameters of the second model include characteristics of the patient selected from the group of sex, race, and smoking history.

17. The method of claim 12 wherein the output further provides relative significance of the body composition measurements in influencing the health assessment value.

18. The method of claim 12 wherein the computerized analysis image tools output physical measurements.

19. The method of claim 18 wherein the output further provides images depicting the physical measurements.

20. The method of claim 12 wherein the body composition measurements are selected from the group consisting of: bone density, fat classification, area, and density, muscle bulk and density, liver volume and density, spleen volume, and aortic plaque burden.
